(19) United States
(12) Patent Application Publication
(10) Pub. No.: US 2021/0174476 A1
(43) Pub. Date: Jun. 10, 2021

(54) METHOD AND SYSTEM FOR PROVIDING BLUR FILTERING TO EMPHASIZE FOCAL REGIONS OR DEPTHS IN ULTRASOUND IMAGE DATA

(71) Applicant: GE Precision Healthcare LLC, Milwaukee, WI (US)

(72) Inventor: Ole Hvidsten Blytt, Toensberg (NO)

(21) Appl. No.: 16/705,704

(22) Filed: Dec. 6, 2019

(51) Int. Cl. G06T 5/00; G06T 7/70; G06T 7/90; G06T 7/00; G06T 7/50

(52) U.S. Cl. CPC G06T 5/003; G06T 7/70; G06T 7/90; G06T 2207/10028; G06T 7/50; G06T 2200/24; G06T 2207/10136; G06T 7/0012

(57) ABSTRACT

A system and method for emphasizing a focal region or depth in a two-dimensional (2D) ultrasound image, such as a 2D color flow image and/or a volume rendering of three-dimensional (3D) and/or four-dimensional (4D) ultrasound image data, by performing blur filtering on image data outside of the focal region or depth is provided. The method may include presenting, by at least one processor, a 2D ultrasound image of an object at a display system. The method may include receiving, by the at least one processor, an identification of a focal area of interest within the 2D ultrasound image. The method may include performing, by the at least one processor, blur filtering on image data of the 2D ultrasound image outside of the identified focal area of interest. The method may include presenting, by the at least one processor, the 2D ultrasound image having the blur filtering at the display system.

### FIELD

Certain embodiments relate to ultrasound imaging. More specifically, certain embodiments relate to a method and system for emphasizing a focal region or depth in ultrasound image data by performing blur filtering on image data outside of the focal region or depth, such as in a two-dimensional (2D) color flow image and/or a volume rendering of three-dimensional (3D) and/or four-dimensional (4D) ultrasound image data.

### BACKGROUND

Ultrasound imaging is a medical imaging technique for imaging organs and soft tissues in a human body. Ultrasound imaging uses real time, non-invasive high frequency sound waves to produce two-dimensional (2D), three-dimensional (3D), and/or four-dimensional (4D) (i.e., real-time/continuous 3D images) images.

Ultrasound imaging is a valuable, non-invasive tool for diagnosing various medical conditions. Acquired ultrasound data may be analyzed and/or processed to detect anatomical structures evaluated by a medical professional to perform the diagnosis. In cases where the ultrasound image is a 2D color flow image or a volume rendering of 3D or 4D image data, improvements to expedite a review process of the clinician, assist in reporting examination results, enhance training, and the like would be desirable.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for emphasizing a focal region or depth in a two-dimensional (2D) ultrasound image, such as a 2D color flow ultrasound image and/or a volume rendering of three-dimensional (3D) and/or four-dimensional (4D) ultrasound image data, by performing blur filtering on image data outside of the focal region or depth, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to emphasize a focal region or depth in a 2D ultrasound image, such as a 2D color flow image and/or a volume rendering of 3D and/or 4D ultrasound image data, in accordance with various embodiments.

FIG. 2 is a block diagram of an exemplary medical workstation that is operable to emphasize a focal region or depth in a 2D ultrasound image, such as a 2D color flow image and/or a volume rendering of 3D and/or 4D ultrasound image data, in accordance with various embodiments.

FIG. 3 is a display of an object depicted in a volume rendering of 3D and/or 4D image data, in accordance with various embodiments.

FIG. 4 is an exemplary display of a volume rendering of 3D and/or 4D ultrasound image data having blur filtering applied to image data outside of a focal area, in accordance with various embodiments.

FIG. 5 is an exemplary display of a volume rendering of 3D and/or 4D ultrasound image data having blur filtering applied to image data outside of a focal area, in accordance with various embodiments.

FIG. 6 is an exemplary display of a 2D color flow image having blur filtering applied to image data outside of a focal area, in accordance with exemplary embodiments.

FIG. 7 is a flow chart illustrating exemplary steps that may be utilized for emphasizing a focal region or depth in a 2D ultrasound image, such as a 2D color flow image and/or a volume rendering of 3D and/or 4D ultrasound image data, in accordance with exemplary embodiments.

### DETAILED DESCRIPTION

Certain embodiments may be found in a method and system for emphasizing a focal region or depth in a two-dimensional (2D) ultrasound image, such as a 2D color flow image and/or a volume rendering of three-dimensional (3D) and/or four-dimensional (4D) ultrasound image data. Various embodiments have the technical effect of emphasizing a focal region or depth in a 2D ultrasound image by performing blur filtering on image data outside of the focal region or depth.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

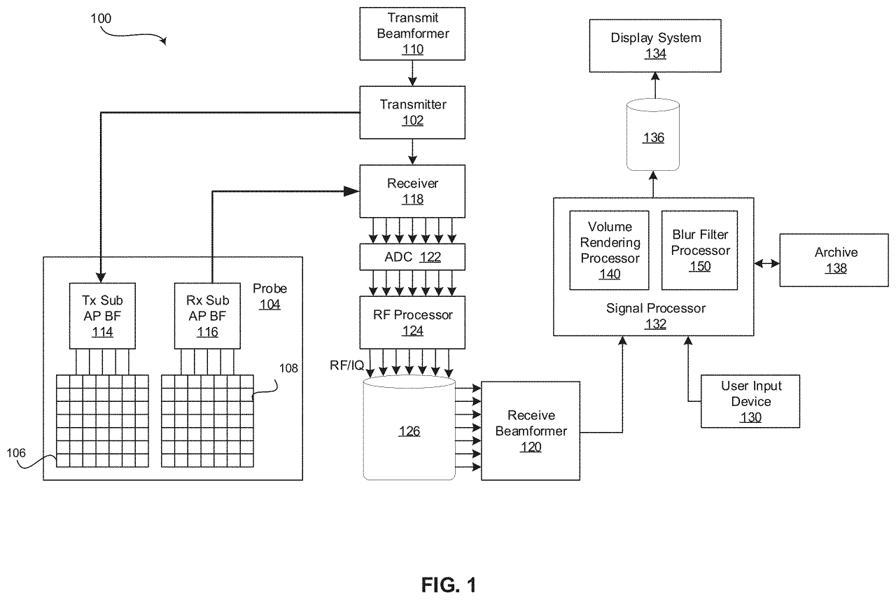

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to emphasize a focal region or depth in a 2D ultrasound image, such as a 2D color flow image and/or a volume rendering of 3D and/or 4D ultrasound image data, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, and an archive 138.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, initiate automated image analysis functionality, select a focal region, select a focal depth, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, a touch pad, a trackball, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display. In various embodiments, blur filtered 2D color flow images and/or volume renderings of 3D or 4D ultrasound image data may be displayed in response to a directive identifying a focal area of interest received via the user input device 130.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a volume rendering processor 140 and a blur filter processor 150. The signal processor 132 may be capable of receiving input information from a user input device 130 and/or archive 138, generating an output displayable by a display system 134, and manipulating the output in response to input information from a user input device 130, among other things. The signal processor 132, including the volume rendering processor 140 and the blur filter processor 150, may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a volume rendering processor 140 that comprises suitable logic, circuitry, interfaces and/or code that may be operable to perform volume rendering on 3D and/or 4D volumes. The volume rendering processor 140 may be used to generate and present volume renderings (e.g., 2D projections) of the volumetric (e.g., 3D and/or 4D) datasets. In this regard, rendering a 2D projection of a 3D and/or 4D dataset may comprise setting or defining a perception angle in space relative to the object being displayed, and then defining or computing necessary information (e.g., opacity and color) for every voxel in the dataset. This may be done, for example, using suitable transfer functions for defining RGBA (red, green, blue, and alpha) value for every voxel. The resulting volume rendering may include a depth map correlating a depth value to each pixel in the 2D projection.

In an exemplary embodiment, the volume rendering processor 140 may be configured to present the volume rendering at the display system 134 and/or may store the volume rendering at archive 138 and/or any suitable storage medium. The volume rendering may be provided to the blur filter processor 150 for (1) post-processing to emphasize a selected focal area, and (2) presentation at the display system 134, as described below.

The signal processor 132 may include a blur filter processor 150 that comprises suitable logic, circuitry, interfaces and/or code that may be operable to post-process and present 2D ultrasound images, such as 2D color flow images and/or volume renderings, to emphasize a selected focal area within the 2D ultrasound image. For example, the selected focal area may be a focal region defined by a position of a cursor manipulated over the 2D ultrasound image. As another example, the selected focal area may be a focal depth selected via the user input device 130. In another example, the selected focal area may be automatically selected by the blur filter processor 150 to highlight structure automatically identified by artificial intelligence image analysis algorithms. The blur filter processor 150 may be operable to receive the 2D ultrasound image, receive a user and/or automated focal area selection, and apply blur filtering to ultrasound image data in the 2D ultrasound image outside of the selected focal area. For example, the blur filter processor 150 may perform blur filtering by applying a box linear filter (i.e., box blur) such that each pixel in the non-focal area has a value equal to an average value of its neighboring pixels to provide the bokeh effect. As another example, the blur filter processor 150 may perform blur filtering by applying Gaussian blur or any suitable blur filtering to de-emphasize areas in the 2D ultrasound image outside of the focal area.

In a representative embodiment, the blur filter processor 150 may be operable to dynamically update the blur filtering in response to changes in the focal area selection. For example, the focal area selection may correspond to a pre-defined shape, such as a circle, box, or any suitable shape, surrounding a cursor. The size of the focal region surrounding the cursor may be a default size or a size selectable and/or adjustable by a user, for example. The focal region surrounding the cursor may be kept in focus by the blur filtering processor 150 while the region outside of the focal region is blurred (i.e., out of focus) by the blur filter processor 150. The focal region may be dynamically updated by the blur filter processor 150 as the cursor is moved about the 2D ultrasound image. As another example, the focal area selection may correspond with a depth selection provided by the user input device 130, such as by scrolling a scroll wheel of a computer mouse, rotating a rotary encoder, manipulating a trackball, selecting a depth from a drop down menu or list of depth options, and the like. The blur filter processor 150 may be operable to dynamically update the blur filtering based on the currently selected depth as a user changes or scrolls through the depth of field such that only the structure at the selected depth remains in focus while structure at depths other than the selected depth is blur filtered by the blur filter processor 150.

In various embodiments, the blur filter processor 150 may comprise suitable logic, circuitry, interfaces and/or code that may be operable to automatically analyze 2D ultrasound images to identify, segment, diagnose, and/or the like structures depicted in the volume renderings. The blur filter processor 150 may include, for example, artificial intelligence image analysis algorithms, one or more deep neural networks (e.g., a convolutional neural network such as u-net) and/or may utilize any suitable form of artificial intelligence image analysis techniques or machine learning processing functionality configured to provide the automated analysis feature(s) and/or tool(s). Additionally and/or alternatively, the artificial intelligence image analysis techniques or machine learning processing functionality configured to provide the automated analysis feature(s) and/or tool(s) may be provided by a different processor or distributed across multiple processors at the ultrasound system 100, a medical workstation 200, and/or a remote processor communicatively coupled to the ultrasound system 100 and/or medical workstation 200. For example, one or more of the image analysis tools may be provided as a deep neural network that may be made up of, for example, an input layer, an output layer, and one or more hidden layers in between the input and output layers. Each of the layers may be made up of a plurality of processing nodes that may be referred to as neurons. For example, one or more of the image analysis tools may include an input layer having a neuron for each pixel or a group of pixels from a 2D ultrasound image of an anatomical structure. The output layer may have a neuron corresponding to a plurality of pre-defined anatomical structures. As an example, if performing an ultrasound-based heart examination, the output layer may include neurons for a mitral valve, aortic valve, ventricle chambers, atria chambers, septum, papillary muscle, inferior wall, and/or any suitable heart structure. Other medical imaging procedures may utilize output layers that include neurons for nerves, vessels, bones, organs, tissue, or any suitable structure. Each neuron of each layer may perform a processing function and pass the processed 2D ultrasound image information to one of a plurality of neurons of a downstream layer for further processing. As an example, neurons of a first layer may learn to recognize edges of structure in the 2D ultrasound image. The neurons of a second layer may learn to recognize shapes based on the detected edges from the first layer. The neurons of a third layer may learn positions of the recognized shapes relative to landmarks in the 2D ultrasound image. The processing performed by the deep neural network may identify anatomical structures, the location of the structures, and abnormalities of the anatomical structures in the 2D ultrasound images with a high degree of probability. In various embodiments, the blur filter processor 150 may select the focal area based on locations of anatomical structures having detected abnormalities. For example, a focal region or depth may be selected to emphasize any abnormalities detected by the image analysis tools. The blur filter processor 150 may apply blur filtering to areas outside of the focal area depicting the structure abnormalities.

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as 2D color flow images, volume renderings, blur filtered 2D color flow images, blur filtered volume renderings, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores 2D ultrasound images, 3D and/or 4D volumes, volume renderings generated by the volume rendering processor 140, instructions for performing volume rendering, blur filtered 2D ultrasound images, instructions for identifying abnormalities in 2D ultrasound images, and/or instructions for performing blur filtering, among other things.

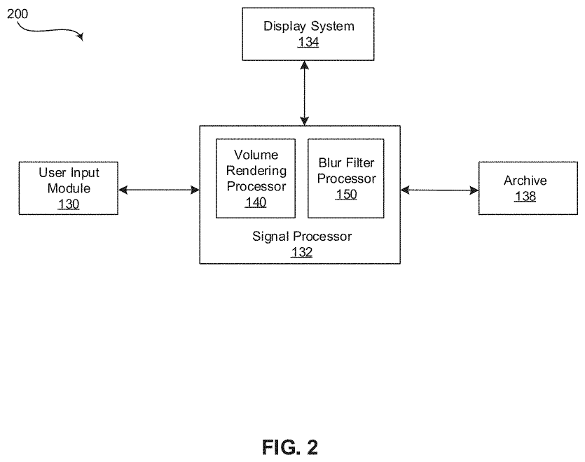

FIG. 2 is a block diagram of an exemplary medical workstation 200 that is operable to emphasize a focal region or depth in a 2D ultrasound image, such as a 2D color flow image and/or a volume rendering of 3D and/or 4D ultrasound image data, in accordance with various embodiments. In various embodiments, components of the medical workstation 200 may share various characteristics with components of the ultrasound system 100, as illustrated in FIG. 1 and described above. Referring to FIG. 2, the medical workstation 200 comprises a display system 134, a signal processor 132, an archive 138, and a user input device 130, among other things. Components of the medical workstation 200 may be implemented in software, hardware, firmware, and/or the like. The various components of the medical workstation 200 may be communicatively linked. Components of the medical workstation 200 may be implemented separately and/or integrated in various forms. For example, the display system 134 and the user input device 130 may be integrated as a touchscreen display.

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as 2D color flow images, volume renderings, blur filtered 2D color flow images, blur filtered volume renderings, and/or any suitable information.

The signal processor 132 may be one or more central processing units, microprocessors, microcontrollers, and/or the like. The signal processor 132 may be an integrated component, or may be distributed across various locations, for example. The signal processor 132 comprises a volume rendering processor 140 and a blur filter processor 150, as described above with reference to FIG. 1, and may be capable of receiving input information from a user input device 130 and/or archive 138, generating an output displayable by a display system 134, and manipulating the output in response to input information from a user input device 130, among other things. The signal processor 132, volume rendering processor 140, and/or blur filter processor 150 may be capable of executing any of the method(s) and/or set(s) of instructions discussed herein in accordance with the various embodiments, for example.

The archive 138 may be one or more computer-readable memories integrated with the medical workstation 200 and/or communicatively coupled (e.g., over a network) to the medical workstation 200, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores 2D ultrasound images, 3D and/or 4D volumes, volume renderings generated by the volume rendering processor 140, instructions for performing volume rendering, blur filtered 2D ultrasound images, instructions for identifying abnormalities in 2D ultrasound images, and/or instructions for performing blur filtering, among other things.

The user input device 130 may include any device(s) capable of communicating information from a user and/or at the direction of the user to the signal processor 132 of the medical workstation 200, for example. As discussed above with respect to FIG. 1, the user input device 130 may include a touch panel, button(s), a mousing device, keyboard, rotary encoder, trackball, touch pad, camera, voice recognition, and/or any other device capable of receiving a user directive.

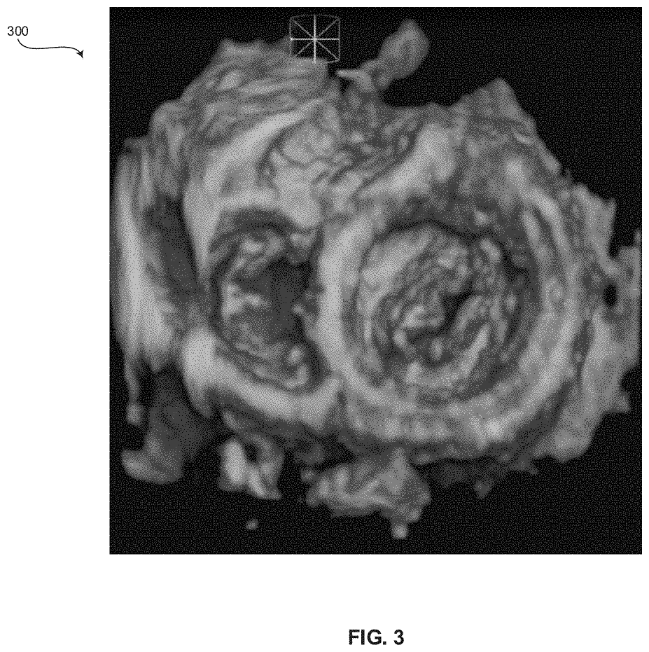

FIG. 3 is a display of an object depicted in a volume rendering 300 of 3D and/or 4D image data, in accordance with various embodiments. Referring to FIG. 3, the volume rendering 300 of 3D and/or 4D image data depicts an object, such as a heart. The volume rendering 300 may be generated from 3D and/or 4D ultrasound data by the volume rendering processor 140, for example, and presented at the display system 134 and/or stored at archive 138 or any suitable data storage medium.

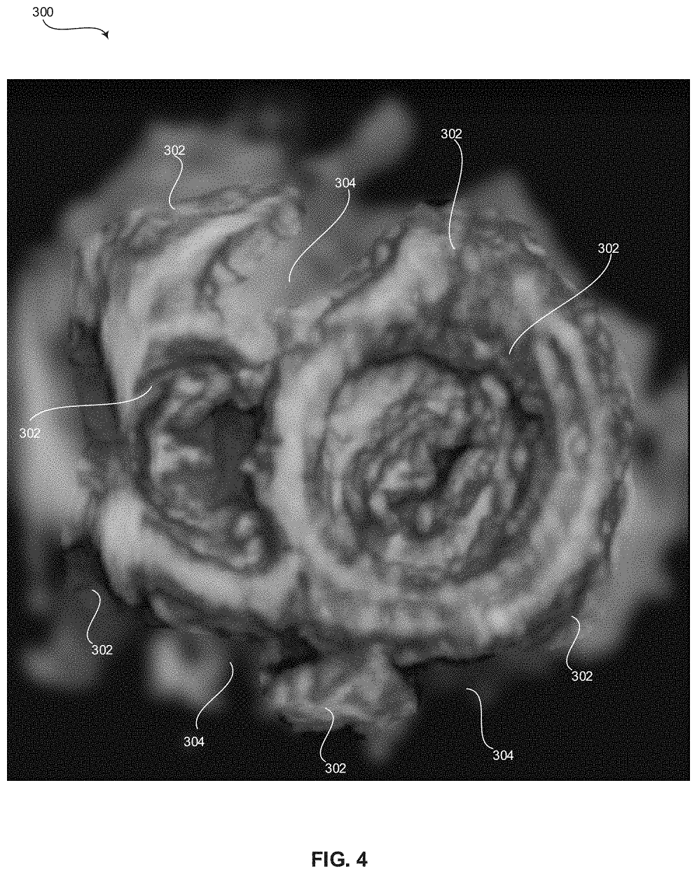

FIG. 4 is an exemplary display of a volume rendering 300 of 3D and/or 4D ultrasound image data having blur filtering 304 applied to image data outside of a focal area 302, in accordance with various embodiments. Referring to FIG. 4, the volume rendering 300 is post-processed to include non-blurred image data 302 in a focal area and blurred image data 304 outside of the focal area. The focal area of non-blurred image data 302 may be selected based on a user input selecting the focal region, a user input selecting the focal depth, and/or artificial intelligence image analysis algorithms selecting a focal region or depth corresponding with detected abnormalities of the imaged structure. The blurred image data 304 may be provided by the blur filter processor 150 to emphasize and/or otherwise call attention to the image data 302 in the selected focal area.

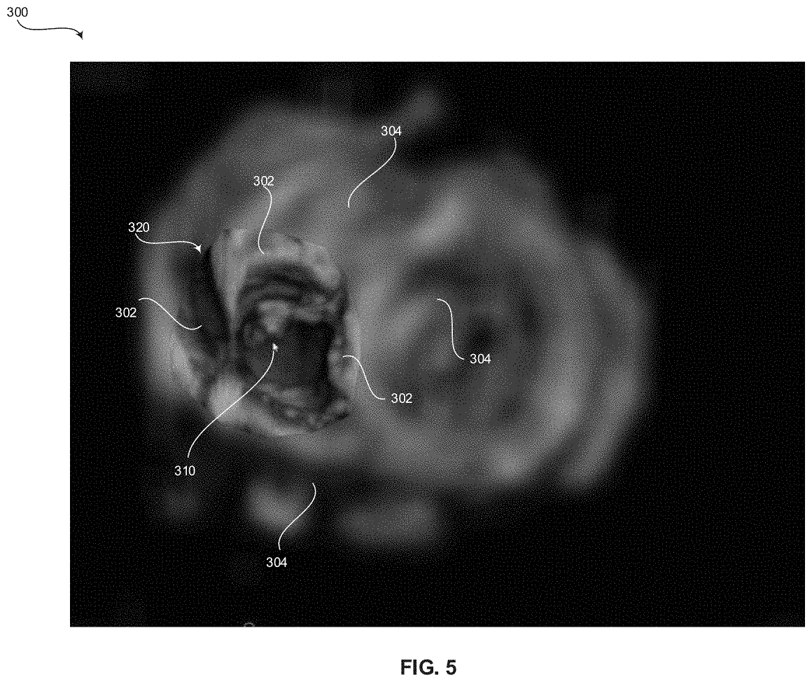

FIG. 5 is an exemplary display of a volume rendering 300 of 3D and/or 4D ultrasound image data having blur filtering 304 applied to image data outside of a focal area 302, in accordance with various embodiments. Referring to FIG. 5, a cursor 310 is positioned over a volume rendering 300 to select a focal region in the volume rendering 300. The volume rendering 300 is post-processed to include non-blurred image data 302 in the focal region surrounding the cursor 310 and blurred image data 304 outside of the focal region. The focal region may be a pre-defined and/or user selected size and shape 320. For example, the focal region of FIG. 5 is a circular shape 320 surrounding the cursor 310. In various embodiments, the shape 320 of the focal region may be box-shaped, oval, or any suitable shape. In various embodiments, the focal region is dynamically updated as the cursor 310 is manipulated about the volume rendering 300. As an example, the focal region surrounding the cursor 310 may be kept in focus by the blur filtering processor 150 while the region outside of the focal region is blurred by the blur filter processor 150 as the cursor 310 is moved around the volume rendering 300 to emphasize the image data surrounding the cursor 310.

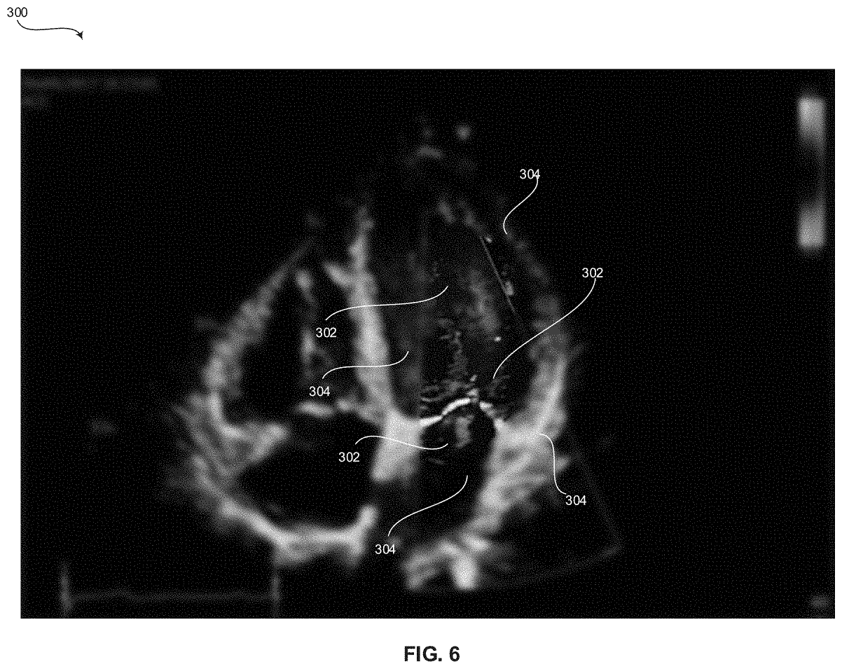

FIG. 6 is an exemplary display of a 2D color flow image 300 having blur filtering 304 applied to image data outside of a focal area 302, in accordance with exemplary embodiments. Referring to FIG. 6, the 2D ultrasound image 300 may be a 2D color flow image having a focal area of non-blurred image data 302 and blurred image data 304 outside of the focal area. For example, the 2D color flow image 300 having the non-blurred 302 and blurred 304 image data may emphasize the non-blurred image data 302 presented to the medical personnel, provided in a report, provided as documentation, and/or the like.

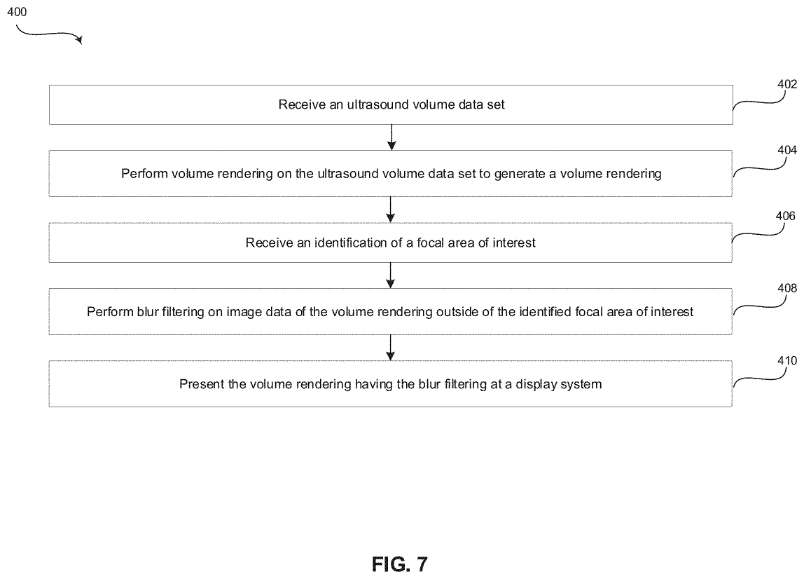

FIG. 7 is a flow chart 400 illustrating exemplary steps 402-410 that may be utilized for emphasizing a focal region or depth 302 in a 2D ultrasound image 300, such as a 2D color flow image and/or a volume rendering of 3D and/or 4D ultrasound image data, in accordance with exemplary embodiments. Referring to FIG. 7, there is shown a flow chart 400 comprising exemplary steps 402 through 410. Certain embodiments may omit one or more of the steps, and/or perform the steps in a different order than the order listed, and/or combine certain of the steps discussed below. For example, some steps may not be performed in certain embodiments. As an example, step 404 may be omitted if the 2D ultrasound image is a 2D color flow image. As a further example, certain steps may be performed in a different temporal order, including simultaneously, than listed below.

At step 402, a signal processor 132 of an ultrasound system 100 or medical workstation 200 may receive an ultrasound volume data set. For example, the ultrasound system 100 may acquire 3D and/or 4D volumes with an ultrasound probe 104. The ultrasound probe 104 may provide the acquired 3D and/or 4D volumes to the signal processor 132. As another example, the signal processor 132 of the medical workstation 200 or ultrasound system 100 may retrieve 3D and/or 4D volumes from an archive 138 or any suitable data storage medium. Alternatively, the signal processor 132 of the ultrasound system 100 or medical workstation 200 may receive a 2D ultrasound image, such as a 2D color flow image or any suitable 2D ultrasound image, and skip to step 406.

At step 404, the signal processor 132 of an ultrasound system 100 or medical workstation 200 may perform volume rendering on the ultrasound volume data set to generate a volume rendering 300. For example, a volume rendering processor 140 of the signal processor 132 of the medical workstation 200 or ultrasound system 100 may render the 3D and/or 4D volumes received at step 402 into a volume rendering 300 for presentation at a display system 134 of the ultrasound system 100 or medical workstation 200.

At step 406, the signal processor 132 of the ultrasound system 100 or workstation 200 may receive an identification of a focal area of interest 302. For example, a blur filtering processor 150 of the signal processor 132 may receive a selected focal area of interest from a user input device 130 or via artificial intelligence image analysis algorithms. As an example, the focal area may be a selected focal region defined by a position of a cursor manipulated via a user input device 130 over the 2D ultrasound image 300. In another example, the focal area may be a focal depth selected via the user input device 130. Additionally and/or alternatively, the focal area may be automatically selected based on imaged structure abnormalities detected by artificial intelligence image analysis algorithms.

At step 408, the signal processor 132 of the ultrasound system 100 or workstation 200 may perform blur filtering on image data 304 of the volume rendering 300 (or any suitable 2D ultrasound image) outside of the identified focal area of interest 302. For example, the blur filtering processor 150 of the signal processor 132 may apply a box linear filter (i.e., box blur), Gaussian blur, and/or any suitable blur filtering to the image data 304 of the volume rendering 300 outside of the focal area 302 to de-emphasize the image data 304 in the volume rendering 300 outside of the focal area 302. The blur filtering may result in non-blurred image data 302 at regions and/or depths within the focal area and blurred image data 304 at regions and/or depths outside of the focal area.

At step 410, the signal processor 132 of the ultrasound system 100 or workstation 200 may present the volume rendering (or any suitable 2D ultrasound image 300) having the blur filtering at a display system 134. In various embodiments, steps 406 through 410 may be dynamically repeated if the focal area is re-defined, such as by a user moving the cursor 310 or scrolling to a different depth.

Aspects of the present disclosure provide a method 400 and system 100, 200 for emphasizing a focal region or depth 302 in a two-dimensional (2D) ultrasound image 300, such as a 2D color flow image and/or a volume rendering of three-dimensional (3D) and/or four-dimensional (4D) ultrasound image data, by performing blur filtering on image data 304 outside of the focal region or depth 302. In accordance with various embodiments, the method 400 may comprise presenting 404, by at least one processor, a 2D ultrasound image 300 of an object at a display system 134. The method 400 may comprise receiving 406, by the at least one processor 132, 150, an identification of a focal area of interest 302 within the 2D ultrasound image 300. The method 400 may comprise performing 408, by the at least one processor 132, 150, blur filtering on image data 304 of the 2D ultrasound image 300 outside of the identified focal area of interest 302. The method 400 may comprise presenting 410, by the at least one processor 132, 150, the 2D ultrasound image 300 having the blur filtering 304 at the display system 134.

In a representative embodiment, the 2D ultrasound image 300 is a 2D color flow image. In an exemplary embodiment, the 2D ultrasound image 300 is a volume rendering, and the method 400 may comprise rendering 404, by the at least one processor 132, 140, a three-dimensional (3D) or four-dimensional (4D) volume to generate the volume rendering 300. In certain embodiments, the focal area of interest 302 is a focal depth selected via a user input device 130. In various embodiments, the focal area of interest 302 is a focal region selected via a user input device 130. In a representative embodiment, the focal region 302 is defined by a position of a cursor 310 manipulated by the user input device 130 over the 2D ultrasound image 300. The focal region 302 may comprise one of a default shape 320 or a user-defined shape 320 surrounding the cursor 310. The focal region 302 may comprise one of a default size or a user-defined size. In an exemplary embodiment, the identification of the focal area of interest 302 may be received by the at least one processor 132, 150 based on at least one abnormality of the object detected by applying at least one artificial intelligence image analysis algorithm. In certain embodiments, the blur filtering 304 may be one of box blur or Gaussian blur.

Various embodiments provide a system 100, 200 for emphasizing a focal region or depth 302 in a 2D ultrasound image 300, such as a 2D color flow image and/or a volume rendering 300 of three-dimensional (3D) and/or four-dimensional (4D) ultrasound image data, by performing blur filtering on image data 304 outside of the focal region or depth 302. The system 100, 200 may comprise at least one processor 132, 140, 150 and a display system 134. The at least one processor 132, 150 may be configured to receive an identification of a focal area of interest 302 within a 2D ultrasound image 300 of an object. The at least one processor 132, 150 may be configured to perform blur filtering on image data 304 of the 2D ultrasound image 300 outside of the identified focal area of interest 302. The display system 134 may be configured to display the 2D ultrasound image 300 having the blur filtering 304.

In an exemplary embodiment, the system 100, 200 may be an ultrasound system 100 or a medical workstation 200. In certain embodiments, the 2D ultrasound image 300 may be a volume rendering. The at least one processor 132, 140 may be configured to render a three-dimensional (3D) or four-dimensional (4D) volume to generate the volume rendering 300. In various embodiments, the system 100, 200 may comprise a user input device 130. The focal area of interest 302 may be a focal depth selected via the user input device 130. In a representative embodiment, the system 100, 200 may comprise a user input device 130. The focal area of interest 302 may be a focal region selected via the user input device 130. The focal region 302 may be defined by a position of a cursor 310 manipulated by the user input device 130 over the 2D ultrasound image 300. The focal region 302 may comprise one of a default shape 320 or a user-defined shape 320 surrounding the cursor 310. The focal region 302 may comprise one of a default size or a user-defined size. In an exemplary embodiment, the at least one processor 132, 150 may be configured to identify the focal area of interest 302 based on at least one abnormality of the object detected by applying at least one artificial intelligence image analysis algorithm. In various embodiments, the blur filtering 304 may be one of box blur or Gaussian blur.

Certain embodiments provide a non-transitory computer readable medium having stored thereon, a computer program having at least one code section. The at least one code section is executable by a machine for causing the machine to perform steps 400. The steps 400 may comprise presenting 404 a 2D ultrasound image 300 of an object at a display system 134. The steps 400 may comprise receiving 406 an identification of a focal area of interest 302 within the 2D ultrasound image 300. The steps 400 may comprise performing 408 blur filtering on image data 304 of the 2D ultrasound image 300 outside of the identified focal area of interest 302. The steps 400 may comprise presenting 410 the 2D ultrasound image 300 having the blur filtering 304 at the display system 134.

In various embodiments, the focal area of interest 302 may be a focal depth selected via a user input device 130. In a representative embodiment, the focal area of interest 302 may be a focal region selected via a user input device 130. The focal region 302 may be defined by a position of a cursor 310 manipulated by the user input device 130 over the 2D ultrasound image 300. The focal region 302 may comprise one of a default shape 320 or a user-defined shape 320 surrounding the cursor 310. The focal region 302 may comprise one of a default size or a user-defined size. In an exemplary embodiment, the identification of the focal area of interest 302 may be based on at least one abnormality of the object detected by applying at least one artificial intelligence image analysis algorithm. In certain embodiments, the blur filtering 304 may be one of box blur or Gaussian blur.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for emphasizing a focal region or depth in a 2D ultrasound image, such as a 2D color flow image and/or a volume rendering of three-dimensional (3D) and/or four-dimensional (4D) ultrasound image data, by performing blur filtering on image data outside of the focal region or depth.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

1. A method comprising:
presenting, by at least one processor, a two-dimensional (2D) ultrasound image of an object at a display system;
receiving, by the at least one processor, an identification of a focal area of interest within the 2D ultrasound image;
performing, by the at least one processor, blur filtering on image data of the 2D ultrasound image outside of the identified focal area of interest; and
presenting, by the at least one processor, the 2D ultrasound image having the blur filtering at the display system.

2. The method of claim 1, wherein the 2D ultrasound image is a 2D color flow image.

3. The method of claim 1, wherein the 2D ultrasound image is a volume rendering, and comprising rendering, by the at least one processor, a three-dimensional (3D) or four-dimensional (4D) volume to generate the volume rendering.

4. The method of claim 1, wherein the focal area of interest is a focal depth selected via a user input device.

5. The method of claim 1, wherein the focal area of interest is a focal region selected via a user input device.

6. The method of claim 5, wherein:
the focal region is defined by a position of a cursor manipulated by the user input device over the 2D ultrasound image,
the focal region comprises one of a default shape or a user-defined shape surrounding the cursor, and
the focal region comprises one of a default size or a user-defined size.

7. The method of claim 1, wherein the identification of the focal area of interest is received by the at least one processor based on at least one abnormality of the object detected by applying at least one artificial intelligence image analysis algorithm.

8. The method of claim 1, wherein the blur filtering is one of box blur or Gaussian blur.

9. A system comprising:
at least one processor configured to:
receive an identification of a focal area of interest within a two-dimensional (2D) ultrasound image of an object; and
perform blur filtering on image data of the 2D ultrasound image outside of the identified focal area of interest; and
a display system configured to display the 2D ultrasound image having the blur filtering.

10. The system of claim 9, wherein the system is an ultrasound system or a medical workstation.

11. The system of claim 9, wherein the 2D ultrasound image is a volume rendering, and wherein the at least one processor is configured to render a three-dimensional (3D) or four-dimensional (4D) volume to generate the volume rendering.

12. The system of claim 9, comprising a user input device, wherein the focal area of interest is a focal depth selected via the user input device.

13. The system of claim 9, comprising a user input device, wherein:
the focal area of interest is a focal region selected via the user input device,
the focal region is defined by a position of a cursor manipulated by the user input device over the 2D ultrasound image,
the focal region comprises one of a default shape or a user-defined shape surrounding the cursor, and the focal region comprises one of a default size or a user-defined size.

14. The system of claim 9, wherein the at least one processor is configured to identify the focal area of interest based on at least one abnormality of the object detected by applying at least one artificial intelligence image analysis algorithm.

15. The system of claim 9, wherein the blur filtering is one of box blur or Gaussian blur.

16. A non-transitory computer readable medium having stored thereon, a computer program having at least one code section, the at least one code section being executable by a machine for causing the machine to perform steps comprising:
presenting a two-dimensional (2D) ultrasound image of an object at a display system;
receiving an identification of a focal area of interest within the 2D ultrasound image;
performing blur filtering on image data of the 2D ultrasound image outside of the identified focal area of interest; and
presenting the 2D ultrasound image having the blur filtering at the display system.

17. The non-transitory computer readable medium of claim 16, wherein the focal area of interest is a focal depth selected via a user input device.

18. The non-transitory computer readable medium of claim 16, wherein:
the focal area of interest is a focal region selected via a user input device,
the focal region is defined by a position of a cursor manipulated by the user input device over the 2D ultrasound image,
the focal region comprises one of a default shape or a user-defined shape surrounding the cursor, and
the focal region comprises one of a default size or a user-defined size.

19. The non-transitory computer readable medium of claim 16, wherein the identification of the focal area of interest is based on at least one abnormality of the object detected by applying at least one artificial intelligence image analysis algorithm.

20. The non-transitory computer readable medium of claim 16, wherein the blur filtering is one of box blur or Gaussian blur.
